(19) United States
(12) Patent Application Publication
(10) Pub. No.: US 2018/0000076 A1
(43) Pub. Date: Jan. 4, 2018

(54) Polypeptides Having N-Acetyl Glucosamine Oxidase Activity

(71) Applicant: Novozymes A/S, Bagsvaerd (DK)

(72) Inventors: Kirk Matthew Schnorr, Holte (DK); Marianne Thorup Cohn, Copenhagen (DK); Lars Henrik Oestergaard, Charlottelund (DK)

(73) Assignee: Novozymes A/S, Bagsvaerd (DK)

(21) Appl. No.: 15/533,523

(22) PCT Filed: Dec. 16, 2015
(86) PCT No.: PCT/EP2015/080014

§ 371 Date: Jun. 6, 2017

(30) Foreign Application Priority Data
Dec. 16, 2014 (EP) 14198289.2

(51) Int. Cl. A01N 37/46; C12N 15/62; C11D 11/00; C11D 3/48; C02F 1/50; A01N 63/00; A61Q 17/00; A61K 38/47; A61K 38/44; A61K 8/64; C12P 21/02; C11D 3/386

(52) U.S. Cl. CPC A01N 37/46; C12P 21/02; C12N 15/62; C11D 11/00; C11D 3/48; C11D 3/386; A01N 63/00; A61Q 17/005; A61K 38/47; A61K 38/44; A61K 8/64; C02F 1/50

(57) ABSTRACT

The present invention relates to polypeptides having N-acetyl glucosamine oxidase activity and polynucleotides encoding the polypeptides. The invention also relates to nucleic acid constructs, vectors, and host cells comprising the polynucleotides as well as methods of producing and using the polypeptides.

### BACKGROUND OF THE INVENTION

### Field of the Invention

The present invention relates to polypeptides having N-acetyl glucosamine oxidase activity, and polynucleotides encoding the polypeptides. The invention also relates to nucleic acid constructs, vectors, and host cells comprising the polynucleotides as well as methods of producing and using the polypeptides. Finally the invention relates to enzymatic composition comprising such polypeptides and capable of killing or inhibiting microbial cells present in laundry, on hard surface, on skin, teeth or mucous membranes; and for preserving food products, cosmetics, paints, coatings.

### Description of the Related Art

Overuse of antibiotics and resultant emergence of microbes resistant to such antibiotics is a serious concern to modern society. Antimicrobial resistance threatens the effective prevention and treatment of an ever-increasing range of infections caused by bacteria, parasites, viruses and fungi. The identification of novel methods to kill bacterial or fungal microorganisms is therefore of increasing interest. The present invention provides such methods.

Hydrogen peroxide is a well described antimicrobial agent. It has long been recognized that some oxidases produce hydrogen peroxide as a bi-product of their oxidation reactions.

Zia et al. 2013 (Brazilian Archives of Biology and Technology 56, 6: 956-961) have shown that the peroxide generating enzyme glucose gxidase (EC 1.1.3.4) has effect as an antimicrobial agent against bacteria such as Staphylococcus aureus and Pastuerella multoida but not Escherichia coli nor the fungi tested, Aspergillus niger or Penicillium notatum.

Patent US2001009664A describes the use of a haloperoxidase system as an effective method of killing microorganisms utilizing a haloperoxidase in the presence of peroxide and a halide. The peroxide can be supplied directly or generated by a peroxide generating enzyme such as the glucose oxidase described above or a lactose oxidase (EC 1.1.3 ×). Patent US2005079165A describes the use of a haloperoxidase system for killing Bacillus spores, a microbial target that is highly resistant to other antimicrobial agents. This haloperoxidase system is reliant on an independent supply of hydrogen peroxide or an enzyme generating peroxide, and a halide, e.g., vanadium halide.

A comparative pairwise global alignment showed that the polypeptide of the present invention shares 72.4% identity to the deduced amino acid sequence of a putative uncharacterized protein from Pyrenophora tritici (accession number SWISSPROT: B2W0N02).

The polypeptide of the present invention shares 25.5% identity to the amino acid sequence of a carbohydrate oxidase from Fusarium graminearum (Heuts et al, 2007. FEBS Lett. 581, 4905-4909).

### SUMMARY OF THE INVENTION

The present invention provides polypeptides having N-acetyl glucosamine oxidase activity and polynucleotides encoding the polypeptides. The polypeptides have bactericidal and fungicidal effects and may have application in disinfection and/or cleaning compositions, e.g., compositions for Cleaning-in-place (CIP) procedures, such as commonly used for cleaning storage tanks, bioreactors, fermenters, mix vessels, pipelines and other equipment used in biotech manufacturing, pharmaceutical manufacturing and food and beverage manufacturing. Furthermore, depending on the concentration of the enzyme or substrate used, a differential effect is observed in which bacteria such as Lactobacillus reuteri is killed but Brewer's yeast (Saccharomyces cerevisae) are unaffected. The polypeptides of the invention (i.e. the enzyme of the invention) can thus be used for control of certain bacteria in yeast fermentations.

The application of the enzyme of the invention, and the bactericidal and fungicidal effects thereof described herein is advantageous in that it does not require a halide nor exogenous peroxide or an additional enzyme that generates additional peroxide. The enzyme of the invention utilizes N-acetyl-glucosamine as a substrate. In this respect, the enzyme of the invention resembles the carbohydrate oxidase from Fusarium graminearum (Heuts et al, 2007. FEBS Lett. 581, 4905-4909) except that the F. graminearum carbohydrate oxidase appears to utilize either N-acetyl glucosamine or N-glucosamine equally while the polypeptide of the invention appear much more active on N-acetyl glucosamine. The present studies (Example 6) indicate that the polypeptide of the invention (DeCOx) is far more efficient at using N-acetyl-D-glucosamine (GlcNAc) than the F. graminearum carbohydrate oxidase (FgCOx). Furthermore, the polypeptide of the invention can also utilize N-acetyl-D-galactosamine (GalNAc) in addition to GlcNAc, a feature that has not been described for any other reported carbohydrate oxidase.

Accordingly, the present invention relates to polypeptides having N-acetyl glucosamine oxidase activity selected from the group consisting of:

(a) a polypeptide having at least 75% sequence identity to the mature polypeptide of SEQ ID NO: 2;

(b) a polypeptide encoded by a polynucleotide that hybridizes under high stringency conditions with (i) the mature polypeptide coding sequence of SEQ ID NO: 1, (ii) the cDNA sequence thereof, or (iii) the full-length complement of (i) or (ii);

(c) a polypeptide encoded by a polynucleotide having at least 75% sequence identity to the mature polypeptide coding sequence of SEQ ID NO: 1 or the cDNA sequence thereof;

(d) a variant of the mature polypeptide of SEQ ID NO: 2 comprising a substitution, deletion, and/or insertion at one or more (e.g., several) positions; and

(e) a fragment of the polypeptide of (a), (b), (c), or (d) that has N-acetyl glucosamine oxidase activity.

The present invention also relates to polypeptides comprising a catalytic domain selected from the group consisting of:

(a) a catalytic domain having at least 75% sequence identity to amino acids 154 to 632 of SEQ ID NO: 2;

(b) a catalytic domain encoded by a polynucleotide that hybridizes under high stringency conditions with (i) nucleotides 509 to 1945 of SEQ ID NO: 1, (ii) the cDNA sequence thereof, or (iii) the full-length complement of (i) or (ii);

(c) a catalytic domain encoded by a polynucleotide having at least 75% sequence identity to nucleotides 509 to 1945 of SEQ ID NO: 1 or the cDNA sequence thereof;

(d) a variant of amino acids 20 to 632 of SEQ ID NO: 2 comprising a substitution, deletion, and/or insertion at one or more (e.g., several) positions; and

(e) a fragment of the catalytic domain of (a), (b), (c), or (d) that has N-acetyl glucosamine oxidase activity.

The present invention also relates to polypeptides comprising a binding domain selected from the group consisting of:

(a) a binding domain having at least 75% sequence identity to amino acids 28 to 71 and/or 101 to 144 of SEQ ID NO: 2;

(b) a binding domain encoded by a polynucleotide that hybridizes under high stringency conditions with (i) nucleotides 82 to 262 and 350 to 481 of SEQ ID NO: 1, (ii) the cDNA sequence thereof, or (iii) the full-length complement of (i) or (ii);

(c) a binding domain encoded by a polynucleotide having at least 75% sequence identity to nucleotides 82 to 262 and 350 to 481 of SEQ ID NO: 1 or the cDNA sequence thereof;

(d) a variant of amino acids 20 to 632 of SEQ ID NO: 2 comprising a substitution, deletion, and/or insertion at one or more (e.g., several) positions; and (e) a fragment of the binding domain of (a), (b), (c), or (d) that has chitin or peptidoglycan binding activity.

The present invention also relates to polynucleotides encoding the polypeptides of the present invention; nucleic acid constructs; recombinant expression vectors; recombinant host cells comprising the polynucleotides; and methods of producing the polypeptides.

The present invention also relates to a composition comprising the polypeptide of the invention and to methods of cleaning and/or disinfecting comprising applying the composition.

### BRIEF DESCRIPTION OF THE SEQUENCES

SEQ ID NO: 1 shows the nucleotide sequence and deduced amino acid sequence of the Didymella exitialis DeCOx gene. The coding sequence is 1948 bp including the stop codon and is interrupted by one intron of 49 bp (nucleotides 142 to 190).

SEQ ID NO: 2 shows the amino acid sequence of the Didymella exitialis N-acetyl-D-glucosamine oxidase (DeCOx).

SEQ ID NO: 3 shows primer DeCOx-F

SEQ ID NO: 4 shows primer DeCOx-R

SEQ ID NO: 5 shows the amino acid sequence of a carbohydrate oxidase from Fusarium graminearum (FgCOx)

SEQ ID NO: 6 shows primer FgCOx-F

SEQ ID NO: 7 shows primer FgCOx-R

### DEFINITIONS

N-acetyl glucosamine oxidase: The term “N-acetylglucosamine oxidase” means a carbohydrate oxidase capable of oxidizing N-acetyl-D-glucosamine in the 1 position. The enzyme is structurally related to carbohydrate oxidases (EC 1.1.3.×) that oxidizes monosaccharides and, the terminal sugar di, tri, tetra and pentasaccarides, with concomitant reduction of molecular oxygen to hydrogen peroxide.

For purposes of the present invention, N-acetyl glucosamine oxidase activity is determined according to the procedure described in Example 6. In one aspect, the polypeptides of the present invention have at least 20%, e.g., at least 40%, at least 50%, at least 60%, at least 70%, at least 80%, at least 90%, at least 95%, or at least 100% of the N-acetyl glucosamine oxidase activity of the mature polypeptide of SEQ ID NO: 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “antimicrobial activity” indicates the polypeptides ability to kill microorganisms or inhibit their growth.^{[1]} The antimicrobial activity may be assessed with any of the methods in Examples 6, 7, 8, or 9. In one aspect, the polypeptides of the present invention have at least 20%, e.g., at least 40%, at least 50%, at least 60%, at least 70%, at least 80%, at least 90%, at least 95%, or at least 100% of the antimicrobial activity of the mature polypeptide of SEQ ID NO: 2.

Biofilm: A biofilm is any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from planktonic bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community.

Binding domain: The term “binding domain” in the context of the present invention means a CBM18 binding domain preferably comprises or consists of amino acids 28 to 71 and to 101 to 144 of SEQ ID NO: 2 or an allelic variant thereof; or is a fragment thereof having chitin or peptidoglycan binding activity.

Catalytic domain: The term “catalytic domain” means the region of an enzyme containing the catalytic machinery of the enzyme having N-acetyl-D-glucosamine activity. The catalytic domain of the present invention is preferably an AA7 oxidase domain.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cleaning-in-place: “Cleaning-in-place” or “CIP” is a method for cleaning the interior surfaces of process equipment or tanks without dismantling the equipment. Process equipment can be processing tanks, storage tanks, pipelines, tubing, heat-exchangers, homogenizers, centrifuges, evaporators, extruders, coolers, storage tanks, sieves, hydroclones, filter units and filter membranes. CIP can also be used in road tankers transporting liquid food such as milk or beer, or in equipment used in slaughter houses.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has N-acetyl glucosamine oxidase activity. In one aspect, a fragment contains at least 310 amino acid residues (e.g., amino acids 20 to 329 of SEQ ID NO: 2), at least 300 amino acid residues (e.g., amino acids 20 to 319 of SEQ ID NO: 2), or at least 290 amino acid residues (e.g., amino acids 20 to 309 of SEQ ID NO: 2).

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 20 to 632 of SEQ ID NO: 2. Amino acids 1 to 19 of SEQ ID NO: 2 are a signal peptide. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having N-acetyl glucosamine oxidase activity. In one aspect, the mature polypeptide coding sequence is nucleotides 58 to 1945 of SEQ ID NO: 1 or the cDNA sequence thereof. Nucleotides 1 to 57 of SEQ ID NO: 1 encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the —nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the —nobrief option) is used as the percent identity and is calculated as follows:

(Identical Deoxyribonucleotides×100)/(Length of Alignment−Total Number of Gaps in Alignment)

Surface: The term “surface” as used herein relates to any surface which may act as support for growth of microbes, such as bacteria, and fungi. The surface may be covered by a biofilm layer. Examples of surfaces may be any hard surface such as metal, plastics, rubber, board, glass, wood, paper, concrete, rock, marble, gypsum and ceramic materials which optionally are coated, e.g., with paint, enamel etc.; or any soft surface such as fibres of any kind (yarns, textiles, vegetable fibres, rock wool, hair etc.); or porous surfaces; skin (human or animal); keratinous materials (nails etc.). The hard surface can be present in a process equipment member of a cooling tower, a water treatment plant, a dairy processing plant, a food processing plant, a chemical or pharmaceutical process plant. The porous surface can be present in a filter, e.g. a membrane filter. Accordingly, the composition and the method according to the present invention are also useful in a conventional cleaning-in-place (CIP) system.

Stringency conditions: The term “very low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 45° C.

The term “low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 50° C.

The term “medium stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 55° C.

The term “medium-high stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 60° C.

The term “high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 65° C.

The term “very high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 70° C.]

Subsequence: The term “subsequence” means a polynucleotide having one or more (e.g., several) nucleotides absent from the 5′ and/or 3′ end of a mature polypeptide coding sequence; wherein the subsequence encodes a fragment having N-acetyl glucosamine oxidase activity.

### DETAILED DESCRIPTION OF THE INVENTION

Polypeptides having N-Acetyl Glucosamine Oxidase Activity

In an embodiment, the present invention relates to polypeptides having a sequence identity to the mature polypeptide of SEQ ID NO: 2 of e.g., at least 75%, at least 80%, at least 85%, at least 90%, at least 91%, at least 92%, at least 93%, at least 94%, at least 95%, at least 96%, at least 97%, at least 98%, at least 99%, or 100%, which have N-acetyl glucosamine oxidase activity. In one aspect, the polypeptides differ by up to 10 amino acids, e.g., 1, 2, 3, 4, 5, 6, 7, 8, 9, or 10, from the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 70% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 75% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 80% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 85% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 90% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 95% of the N-acetyl glucosamine oxidase activity of the mature polypeptide of SEQ ID NO: 2.

In a particular embodiment the invention relates to polypeptides having a sequence identity to the mature polypeptide of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least 100% of the N-acetyl glucosamine oxidase activity of the mature polypeptide of SEQ ID NO: 2.

In an embodiment, the polypeptide has been isolated. A polypeptide of the present invention preferably comprises or consists of the amino acid sequence of SEQ ID NO: 2 or an allelic variant thereof; or is a fragment thereof having N-acetyl glucosamine oxidase activity. In another aspect, the polypeptide comprises or consists of the mature polypeptide of SEQ ID NO: 2. In another aspect, the polypeptide comprises or consists of amino acids ? to ? of SEQ ID NO: 2.

In another embodiment, the present invention relates to a polypeptide having N-acetyl glucosamine oxidase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the cDNA sequence thereof], or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 1 or a subsequence thereof, as well as the polypeptide of SEQ ID NO: 2 or a fragment thereof may be used to design nucleic acid probes to identify and clone DNA encoding polypeptides having N-acetyl glucosamine oxidase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having N-acetyl glucosamine oxidase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1 or the cDNA sequence thereof.

In another embodiment, the present invention relates to an polypeptide having N-acetyl glucosamine oxidase activity encoded by a polynucleotide having a sequence identity to the mature polypeptide coding sequence of SEQ ID NO: 1 or the cDNA sequence thereof of at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, AlaNal, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for N-acetyl glucosamine oxidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Sources of Polypeptides having N-Acetyl Glucosamine Oxidase Activity

A polypeptide having N-acetyl glucosamine oxidase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In an aspect, the polypeptide is a Didymella polypeptide, e.g., a polypeptide obtained from Didymella exitialis.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Catalytic Domains

In one embodiment, the present invention also relates to catalytic domains having a sequence identity to amino acids 154 to 632 of SEQ ID NO: 2 of at least 75%, at least 80%, at least 85%, at least 90%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 154 to 632 of SEQ ID NO: 2.

The catalytic domain preferably comprises or consists of amino acids 154 to 632 of SEQ ID NO: 2 or an allelic variant thereof; or is a fragment thereof having N-acetyl glucosamine oxidase activity.

In another embodiment, the present invention also relates to catalytic domains encoded by polynucleotides that hybridize under high stringency conditions (as defined above) with (i) the nucleotides 509 to 1945 of SEQ ID NO: 2, (ii) the cDNA sequence thereof, or (iii) the full-length complement of (i) or (ii) (Sambrook et al., 1989, supra).

In another embodiment, the present invention also relates to catalytic domains encoded by polynucleotides having a sequence identity to nucleotides 509 to 1945 of SEQ ID NO: 1 or the cDNA sequence thereof of at least 75%, at least 80%, at least 85%, at least 90%, at least least 95%, at least 96%, at least 97%, at least 98%, at least 99%, or 100%.

The polynucleotide encoding the catalytic domain preferably comprises or consists of nucleotides 509 to 1945 of SEQ ID NO: 1.

In another embodiment, the present invention also relates to catalytic domain variants of amino acids 509 to 1945 of SEQ ID NO: 2 comprising a substitution, deletion, and/or insertion at one or more (e.g., several) positions. In one aspect, the number of amino acid substitutions, deletetions and/or insertions introduced into the sequence of amino acids 154 to 632 of SEQ ID NO: 2 is up to 10, e.g., 1, 2, 3, 4, 5, 6, 8, 9, or 10.

### Binding Domains

In one embodiment, the present invention also relates to binding domains having a sequence identity to amino acids 28 to 71 and/or amino acids 101 to 144 of SEQ ID NO: 2 of at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the binding domains comprise amino acid sequences that differ by up to 10 amino acids, e.g., 1, 2, 3, 4, 5, 6, 7, 8, 9, or 10, from amino acids 28 to 71 and/or amino acids 101 to 144 of SEQ ID NO: 2.

The binding domain preferably comprises or consists of amino acids 28 to 71 and/or amino acids 101 to 144 of SEQ ID NO: 2 or an allelic variant thereof; or is a fragment thereof having chitin or peptidoglycan binding activity.

In another embodiment, the present invention also relates to binding domains encoded by polynucleotides that hybridize under very low stringency conditions, low stringency conditions, medium stringency conditions, medium-high stringency conditions, high stringency conditions, or very high stringency conditions (as defined above) with (i) the nucleotides 82 to 262 and/or 350 to 481of SEQ ID NO: 1, (ii) the cDNA sequence thereof, or (iii) the full-length complement of (i) or (ii) (Sambrook et al., 1989, supra).

In another embodiment, the present invention also relates to binding domains encoded by polynucleotides having a sequence identity to nucleotides 82 to 262 and/or nucleotides 350 to 481 of SEQ ID NO: 1 of at least 65%, e.g., at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

The polynucleotide encoding the binding domain preferably comprises or consists of nucleotides 82 to 262 and/or nucleotides 350 to 481 of SEQ ID NO: 1.

In another embodiment, the present invention also relates to binding domain variants of amino acids 28 to 71 and/or 101 to 144 of SEQ ID NO: 2 comprising a substitution, deletion, and/or insertion at one or more (e.g., several) positions. In one aspect, the number of amino acid substitutions, deletions and/or insertions introduced into the sequence of amino acids 28 to 71 and/or amino acids 101 to 144 of SEQ ID NO: 2 is up to 10, e.g., 1, 2, 3, 4, 5, 6, 8, 9, or 10.

### Polynucleotides

The present invention also relates to polynucleotides encoding a polypeptide, a catalytic domain, or binding domain of the present invention, as described herein. In an embodiment, the polynucleotide encoding the polypeptide, catalytic domain, or binding domain of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Didymella, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The host cell may also be a eukaryote, such as a mammalian, insect, plant, or fungal cell.

Preferably, the host cell is a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series Nov. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

Preferably, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Preferably, the fungal cell is an Aspergillus cell and more preferably an Aspergillus niger, or an Aspergillus oryzae cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Didymella cell. In another aspect, the cell is a Didymella exitialis cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

### Detergent Compositions

The polypeptide of present invention may be incorporated in a detergent composition for application in laundry and dish wash.

The invention provides a method for antimicrobial treatment of microorganisms or viruses present on laundry and/or in a liquid used for soaking, washing or rinsing the laundry, e.g. in a washing machine.

In one embodiment, the invention is directed to detergent compositions comprising an enzyme of the present invention in combination with one or more additional cleaning composition components, such as e.g., a surfactant. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to an ADW (Automatic Dish Wash) composition comprising an enzyme of the present invention in combination with one or more additional ADW composition components, such as e.g., a surfactant. The choice of additional components is within the skill of the artisan and includes conventional ingredients, including the exemplary non-limiting components set forth below.

When applied in laundry and/or ADW the treatment with the polypeptide of the present invention also has an antimicrobial effect on biofilm inside a washing machine and an ADW machine. The polypeptide of the present invention may thus be applied in a method for preventing or reducing biofilm formation in a washing machine and/or an ADW machine. The polypeptide of the present invention may thus be applied in a method for preventing or reducing malodor formation in a washing machine and/or an ADW machine.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is chosen based on the desired cleaning application, and may include any conventional surfactant(s) known in the art. A suitable non-ionic surfactant may be any of of glycerol derivatives, sorbitan, glucose, sucrose derivatives, fatty acid ethoxylates, fatty acid ethoxylates propoxylates, fatty alcohol ethoxylates, alkyl phenol ethoxylates, fatty alcohol ethoxylates propoxylates, fatty esters of polyalcohol ethoxylates, end-blocked ethoxylates, polypropylene glycols and polyethylene glycols.

The surfactant(s) is typically present at a level of from about 0.1% to 60% by weight, such as about 1% to about 40% by weight of the composition, such as from about 5% to about 30%, including from about 5% to about 15%, or from about 15% to about 20%, or from about 20% to about 25% of the composition.

### Hydrotropes

The detergent composition may contain a hydrotrope. A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants). Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in detergent compositions may be utilized.

### Bleaching Systems

The detergent compositions may contain 0-30% by weight, such as about 1% to about 20%, of a bleaching system. Any bleaching system known in the art for use in detergent compositions may be utilized. Suitable bleaching system components include bleaching catalysts, photobleaches, bleach activators, sources of hydrogen peroxide such as sodium percarbonate, sodium perborates and hydrogen peroxide—urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacety lethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

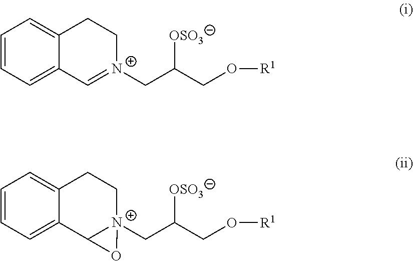

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent compositions of the present invention may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers , hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Enzymes

The detergent additive as well as the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Mannanases: Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

Proteases: Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO095/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Purafect MA®, Purafect Ox®, Purafect Ox®, Puramax®, Properase®, FN2®, FN3®, FN4®, Excellase®, Eraser®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and cutinases: Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases: Suitable amylases which can be used together with the polypeptide of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions N21D+D97N+V128I wherein the variants optionally further comprises a substitution at position 200 and/or a deletion at position 180 and/or position 1851.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme PIus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

The detergent enzyme(s) may be included in a detergent composition by adding separate additives containing one or more enzymes, or by adding a combined additive comprising all of these enzymes. A detergent additive, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. NoS. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in detergent compositions may be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in detergent compositions may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diary) pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

### Laundry Soap Bars

The enzyme composition of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes.

The multi-enzyme co-granule may comprise an enzyme of the invention and (a) one or more enzymes selected from the group consisting of lipases, cellulases, xyloglucanases, perhydrolases, peroxidases, lipoxygenases, laccases, hemicellulases, proteases, cellobiose dehydrogenases, xylanases, phospholipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### Sources of Substrate

N-acetyl-D-glucosamine, the substrate of the enzyme of the invention can be found in several sources. A suitable sources of N-acetyl-D-glucosamine will often be present in environments when the enzyme of the invention can be applied, e.g., in fermentation processes or during cleaning-in-place procedures in food production, e.g. in dairy.

GlcNAc is the monomeric unit of the polymer chitin, which forms the outer coverings of insects and crustaceans. Chitin is a polysaccharide that is highly abundant in nature and consists of beta-1,4 linked N-acetylglucosamine It is the main component of the radulas of mollusks, the beaks of cephalopods, and a major component of the cell walls of most fungi and bacteria. Exochitinase enzymes can be used to liberate N-acetylglucosamine from chitin.

Furthermore, milk from mammals, e.g., cows milk, contains millimolar concentrations of N-acetylglucosamine-1-phosphate which, upon dephosphorylation yields N-actyl-D-glucosamine (Belloque, J., Villamiel, M., López.Fandiño, A., and Olano, A (2001) Food Chemistry 72:407-412). Milk also comprises phosphatase capable of dephosphorylation of N-acetylglucosamine-1-phosphate. So milk residues present in food production sites exposed to cleaning using the polypeptide of the invention, e.g. in dairy, will deliver the substrate (Burton et al. (1988) In: Ultra-high-temperature processing of milk and milk products (pp. 45-76), London and New York: Elsvier Applied Science, Knight et al.(1989) Journal of the Society of Dairy Technology, 42, 81-86).

The composition, such as a detergent composition, a disinfection compositions and/or cleaning compositions comprising the enzyme of the invention may also comprise N-acteylglucosamine, or a source of N-acteylglucosamine. In addition a phosphatase capable of dephosphorylation of N-acetylglucosamine-1-phosphate may be included.

### OTHER USES

### Treatment of Human or Animals

The polypeptide of the invention may be used in a composition for antimicrobial treatment of microorganisms or viruses present on or in human or animal skin, hair, oral cavities, glands, mucous membranes, teeth, eyes, wounds or bruises.

Thus the polypeptide of the invention may be used in a composition useful for disinfection, e.g. treatment of acne or other skin infections, infections in the eye or the mouth, microbial growth on feet, in armpits; teeth (oral care), wounds, bruises and the like. The polypeptide of the invention may be useful for controlling bacterial and/or fungal growth in or on a domestic animal, such as cattle, buffalo, sheep, goats, pigs, turkeys, chickens, roosters, and ducks. The polypeptide having N-acetyl-D-glucosamine oxidase activity may be used in a composition for application to the skin of the domestic animal, e.g., for controlling a disease of the animal, such as mastitis and epidermal infections of the udder.

The composition may be applied by use of an aerosol, liquid, emulsion, gel, slurry, paste or solid comprising the enzyme of the invention.

### Treatment of Cosmetics, Food or Beverages or Other Products

The invention may be useful for preservation of food, beverages, cosmetics such as lotions, creams, gels, ointments, soaps, shampoos, conditioners, antiperspirants, deodorants, mouth wash, contact lens products, foot bath products; enzyme formulations, or food ingredients, and food products, e.g., dairy. The invention may be applied to the unpreserved food, beverages, cosmetics, food ingredients in amounts effective for obtaining the desired antimicrobial effect.

### Treatment of Hard Surfaces and CIP

The present invention provides polypeptide and a method of treatment using the polypeptide, which is useful for antimicrobial treatment of any hard surface as defined earlier. The treatment may be applied for general disinfection purposes, e.g. disinfection of hospital wards, operation rooms, rooms for food processing or other facilities, which require disinfection. The hard surface can also be a process equipment member of a cooling tower, a water treatment plant, a processing plant for dairy, a processing plant for food or food additives, a chemical or pharmaceutical process plant. The hard surface may also be a medical device or a water sanitation equipment. The polypeptide may, through a medium, be brought in contact with the surface in question and should be present in amounts, which is has an antimicrobial effect.

The polypeptide of the invention may be applied in a cleaning-in-place procedure. Cleaning-in-place (CIP) is commonly used for cleaning storage tanks, bioreactors, fermenters, mix vessels, pipelines and other equipment used in biotech manufacturing, pharmaceutical manufacturing and food and beverage manufacturing. Accordingly, the invention provides an antimicrobial method, which is useful in a conventional cleaning-in-place system.

Repeatable reliabile, and effective of cleaning is of the utmost importance in a manufacturing facility. Cleaning procedures are validated to demonstrate that they are effective, reproducible, and under control. In order to adequately clean processing equipment the equipment must be designed with smooth stainless steel surfaces and interconnecting piping that has cleanable joints. The cleaning properties of the cleaning agents must properly interact with the chemical and physical properties of the residues being removed. A typical CIP cycle consists of many steps which often include:
Flush/Pre-rinse with clean water flux, which may be heated up to 60-80° C. and recirculated for a period of time.
Circulation of caustic soda solution at a temperature about 20-80° C. for a period of time.
Intermediate rinsing with clean water flux.
Circulation of acidic solution for a period of time.
Final rinse with clean water flux.
Final air blow.

The use of harsh chemicals in CIP is undesirable and poses a problem to the environment. In the past years CIP methods including the use of enzymes have been developed. Patent application WO97/02753 concerns a solution comprising a protease and a lipase for cleaning-in-place. The solution has been found effective in cleaning process equipment containing residues of milk or burnt milk.

The polypeptide of the present invention may be applied as a disinfectant in such CIP methods. The polypeptide is in particular suitable for application in processing equipment for dairy, as milk residues will provide the substrate for the polypeptide.

### Treatment of a Fermentation Medium

Bacterial contamination in fermentation cultures cause losses in the biofuel production where Lactobacillus species are the predominant contaminant. The polypeptide of the invention may be applied for the control of such contamination, e.g., fungal and/or bacterial contamination in yeast ethanol fermentations. The lactic acid bacteria are susceptible to the DeCOx enzyme at concentrations in which brewer's yeast is not significantly affected, also under conditions of some shit limited oxygen such as in a fermentation process.

As demonstrated in the Examples the polypeptide of the invention may be applied for controlling Lactobacillus growth during the process of fermentation, such as fermentation of starch sources and/or other fermentable sugars, e.g., from sugar cane, corn, sorghum, wheat, barley, potato, cassava, rice, malt, grapes, juice and various fruits, as well as from lignocellulosic sources, such as leaves, wood, bagasse, bran, grass, husks, and seeds. The use of the polypeptide of the invention as a natural biocide in fermentation processes as claimed herein, relates to fermentation processes for the production of 1st and/or 2nd generation fuel ethanol as well as ethanol for human consumption, such as distilled alcohol from cereals and corn, as well as beverages such as beer, wine and the like.

The polypeptide of the invention may also be applied for controlling microbes during the chemical and/or enzymatic hydrolysis of the starch sources and/or lignocellulosic sources during the production of sugars. The sugars may be used e.g., for fermentation, as sweeteners or as raw material for the biochemical industry.

The present invention is further described by the following paragraphs.
1. An polypeptide having N-acetyl-D-glucosamine oxidase activity, selected from the group consisting of:

(a) a polypeptide having at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2;

(b) a polypeptide encoded by a polynucleotide that hybridizes under high stringency conditions with (i) the mature polypeptide coding sequence of SEQ ID NO: 1, (ii) the cDNA sequence thereof, or (iii) the full-length complement of (i) or (ii);

(c) a polypeptide encoded by a polynucleotide having at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1 or the cDNA sequence thereof;

(d) a variant of the mature polypeptide of SEQ ID NO: 2 comprising a substitution, deletion, and/or insertion at one or more positions; and

(e) a fragment of the polypeptide of (a), (b), (c), or (d) that has N-acetyl-D-glucosamine oxidase activity.
2. The polypeptide of paragraph 1, comprising or consisting of SEQ ID NO: 2 or the mature polypeptide of SEQ ID NO: 2.
3. The polypeptide of any of paragraphs 1 or 2, wherein the mature polypeptide is amino acids 20 to 632 of SEQ ID NO: 2.
4. The polypeptide of any of paragraphs 1-3, which is a variant of the mature polypeptide of SEQ ID NO: 2 comprising a substitution, deletion, and/or insertion at one or more positions.
5. The polypeptide of any of paragraphs 1-4, which is a fragment of SEQ ID NO: 2, wherein the fragment has N-acetyl-D-glucosamine oxidase activity.
6. The polypeptide of paragraph 1-5; wherein the polypeptide furthermore has N-acetyl-D-galactosamine activity.
7. An polypeptide comprising a catalytic domain selected from the group consisting of:

(a) a catalytic domain having at least 75% sequence identity to amino acids 154 to 632 of SEQ ID NO: 2;

(b) a catalytic domain encoded by a polynucleotide that hybridizes under high stringency conditions with (i) nucleotides 509 to 1945 of SEQ ID NO: 1, (ii) the cDNA sequence thereof, or (iii) the full-length complement of (i) or (ii);

(c) a catalytic domain encoded by a polynucleotide having at least 75% sequence identity to the catalytic domain of SEQ ID NO: 1 or the cDNA sequence thereof;

(d) a variant of amino acids 20 to 632 of SEQ ID NO: 2 comprising a substitution, deletion, and/or insertion at one or more positions; and

(e) a fragment of the catalytic domain of (a), (b), (c), or (d) that has N-acetyl-D-glucosamine oxidase activity.
8. The polypeptide of paragraph 7, further comprising a binding domain.
9. An polypeptide comprising a binding domain operably linked to a catalytic domain, wherein the binding domain is selected from the group consisting of:

(a) a binding domain having at least 75% sequence identity to amino acids 28 to 71 and/or amino acids 101 to 144 of SEQ ID NO: 2;

(b) a binding domain encoded by a polynucleotide that hybridizes under high stringency conditions with (i) nucleotides 82 to 262 and/or nucleotides 350 to 481 of SEQ ID NO: 1, (ii) the cDNA sequence thereof, or (iii) the full-length complement of (i) or (ii);

(c) a binding domain encoded by a polynucleotide having at least 75% sequence identity to nucleotides 82 to 262 and/or nucleotides 350 to 481 of SEQ ID NO: 1 or the cDNA sequence thereof;

(d) a variant of amino acids 28 to 71 and/or amino acids 101 to 144 of SEQ ID NO: 2 comprising a substitution, deletion, and/or insertion at one or more positions; and

(e) a fragment of (a), (b), (c), (d) or (e) that has chitin or peptidoglycan binding activity.
10. The polypeptide of paragraph 9, wherein the catalytic domain is obtained from an N-acetyl-D-glucosamine oxidase, a carbohydrate oxidase, a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.
11. The polypeptide of any of paragraphs 1-10, which has at least 20%, e.g., at least 40%, at least 50%, at least 60%, at least 70%, at least 80%, at least 90%, at least 95%, or at least 100% of the antimicrobial activity of the mature polypeptide of SEQ ID NO: 2.
12. The polypeptide of paragraph 11, wherein the antimicrobial activity is assessed using a method selected from the methods in Examples 6, 7, 8, or 9.
13. The polypeptide of paragraphs 11-12, wherein the antimicrobial activity is assessed as fungicidal or sporicidal effect towards any of the fungal strains Aspergillus carbonarius BR00732 (CBS 139193) and/or Aspergillus niger strain BR00883 (accession number CBS 139194).
14. A polynucleotide encoding the polypeptide of any of paragraphs 1-13.
15. A nucleic acid construct or expression vector comprising the polynucleotide of paragraph 14 operably linked to one or more control sequences that direct the production of the polypeptide in an expression host.
16. A recombinant host cell comprising the polynucleotide of paragraph 14 operably linked to one or more control sequences that direct the production of the polypeptide.
17. A method of producing the polypeptide of any of paragraphs 1-14, comprising cultivating a cell, which in its wild-type form produces the polypeptide, under conditions conducive for production of the polypeptide.
18. The method of paragraph 17, further comprising recovering the polypeptide.
19. A method of producing a polypeptide having N-acetyl-D-glucosamine oxidase activity, comprising cultivating the host cell of paragraph 16 under conditions conducive for production of the polypeptide.
20. The method of paragraph 19, further comprising recovering the polypeptide.
21. A transgenic plant, plant part or plant cell transformed with a polynucleotide encoding the polypeptide of any of paragraphs 1-13.
22. A method of producing a polypeptide having N-acetyl-D-glucosamine oxidase activity, comprising cultivating the transgenic plant or plant cell of paragraph 21 under conditions conducive for production of the polypeptide.
23. The method of paragraph 22, further comprising recovering the polypeptide.
24. A composition comprising the polypeptide of any of paragraphs 1-13.
25. The composition the proceeding paragraph which further comprises a surfactant.
26. The composition of any of the proceeding paragraphs which is or includes a fermentation broth formulation comprising the polypeptide of any of paragraphs 1-13.
27. A cleaning and/or disinfecting composition comprising one or more polypeptide having N-acetyl-D-glucosamine oxidase activity, and a surfactant.
28. The composition according to paragraph 27, wherein the surfactant is one or more non-ionic surfactants.
29. The composition according to paragraph 28, wherein the non-ionic surfactant is selected from the group consisting of glycerol derivatives, sorbitan, glucose, sucrose derivatives, fatty acid ethoxylates, fatty acid ethoxylates propoxylates, fatty alcohol ethoxylates, alkyl phenol ethoxylates, fatty alcohol ethoxylates propoxylates, fatty esters of polyalcohol ethoxylates, end-blocked ethoxylates, polypropylene glycols and polyethylene glycols.
30. The composition according to any of paragraph 24-29, wherein the surfactant is present in an amount from about 1% to about 40% by weight of the composition, such as from about 5% to about 30%, including from about 5% to about 15%, or from about 15% to about 20%, or from about 20% to about 25% of the composition.
31. The composition according to any of paragraphs 24-30, wherein the composition comprising one or more enzymes capable of dephosphorylation of N-acetylglucosamine-1-phosphate.
32. The composition according to any of paragraphs 24-31, wherein the composition comprising one or more sources of N-acetyl-D-glucosamine.
33. The composition according to any of paragraphs 24-32, wherein the composition comprising one or more sources of N-acetyl-D-galactosamine.
34. The composition according to any of paragraphs 24-33, wherein the composition is a liquid composition.
35. The composition according to any of paragraphs 24-34, which is a laundry detergent composition.
36. The composition according to any of paragraphs 24-35, which composition comprises the polypeptide of any of paragraphs 1-13.
37. A method of cleaning and/or disinfecting comprising applying the composition of any of paragraphs 24-35.
38. The method according to paragraph 37, which is a method of controlling bacterial and/or fungal growth.
39. The method according to paragraphs 37 or 38 which is a method of controlling bacterial and/or fungal growth in a fermentation process.
40. The method according to any of paragraphs 37-39, which is a method of controlling bacterial and/or fungal growth in a yeast fermentation.
41. The method according to any of paragraphs 37-40, which is a method of controlling Lactobacillus growth in a yeast fermentation.
42. The method according to any of paragraphs 37-41, wherein the yeast fermentation is a yeast fermentation for alcohol production.
43. The method according to paragraph 37, for antimicrobial treatment of a hard surface.
44. The method according to paragraph 43, wherein the hard surface is a facility, which requires disinfection.
45. The method according to paragraph 44, wherein the facility is a hospital room, a room for processing of food or food additives, a room for water treatment, a room for paper and/or pulp processing or a room for chemical or pharmaceutical processing.
46. The method according to paragraph 45, wherein the hard surface is process equipment within a cooling tower, a water treatment plant, processing equipment for dairy, a processing plant for food or food additives, a processing plant for paper and/or pulp, a chemical or pharmaceutical process plant.
47. The method according to paragraph 46, wherein the hard surface is a surface of water sanitation equipment.
48. The method according to paragraph 47, wherein the hard surface is a surface of a medical device.
49. A method of controlling bacterial and/or fungal growth, said method comprising applying a polypeptide having enzyme activity and capable of utilizing amino acyl substituted sugars as a substrate for oxidation.
50. A method of controlling bacterial and/or fungal growth, said method comprising applying a polypeptide having N-acetyl-D-glucosamine oxidase activity.

51. The method of paragraph 50, wherein said polypeptide further has N-acetyl-D-galactosamine oxidase activity.
52. The method of any of paragraphs 50-51, which comprises adding a source of N-acetyl-D-glucosamine.
53. The method of any of paragraphs 50-52, which comprises adding a source of N-acetyl-D-galactosamine.
54. The method of any of paragraphs 50-53, which is a method of controlling bacterial and/or fungal growth in a yeast fermentation.
55. The method of any of paragraphs 50-54, wherein the yeast fermentation is a yeast alcohol fermentation.
56. The method of any of paragraphs 50-55, which is a method of controlling bacterial and/or fungal growth in the dairy industry.
57. The method of any of paragraphs 50-56, which is a method of controlling bacterial and/or fungal growth in or on a domestic animal, such as an animal selected from the group consisting of cattle, buffalo, sheep, goats, pigs, turkeys, chickens, roosters, and ducks.
58. The method of any of paragraphs 50-57, which comprises applying the polypeptide having N-acetyl-D-glucosamine oxidase activity to the skin of the domestic animal.
59. The method of any of paragraphs 50-58, which is a method of controlling a disease of a domestic animal, such as a disease selected from the group consisting of mastitis and epidermal infections of the udder.
60. The method of any of paragraphs 50-59, which is a method of for preventing, reducing or removing a biofilm on a surface.
61. The method of any of paragraphs 50-60, wherein the surface comprises a material selected from the group consisting of metal, glass, rubber (natural rubber or synthetic rubber), cotton, plastic, PVC, acrylics, nylon, wood, cotton, acryl, polycarbonate, pvc, polypropylene, enamel, ceramics, earthenware, china or porcelain.
62. The method of any of paragraphs 50-61, wherein the surface comprises a material selected from the group consisting of stainless steel, iron, copper, magnesium, chromium, nickel, aluminium, titanium, molybdenum lead, brass, tin, galvanized material, zinc, or an alloy thereof.
63. The method of any of paragraphs 50-62, wherein the surface is a fabric and/or textile.
64. A method for cleaning and disinfecting a surface at least partly covered by a biofilm layer, the method comprising contacting the biofilm with a composition comprising one or more polypeptide having N-acetyl-D-glucosamine oxidase activity.
65. Use of a carbohydrate oxidase for cleaning or disinfection of a surface said carbohydrate oxidase having activity towards N-acetyl-D-glucosamine and N-acetyl-D-galactosamine.
66. Use of a polypeptide having N-acetyl-D-glucosamine oxidase activity and N-acetyl-D-galactosamine for cleaning or disinfection of a surface.
67. Use according to any of paragraphs 65 or 66 for performing CIP.
68. The method, composition or use according to any of paragraphs 51-67 wherein the polypeptide having N-acetyl-D-glucosamine oxidase activity is a polypeptide having at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2 and/or to amino acids 154 to 632 of SEQ ID NO. 2.

### EXAMPLES

### Strains

The Didymella exitialis fungal strain was isolated from Keldskov forest on Lolland, Denmark in 2011.

### Media and Solutions

Materials

Chemicals used as buffers and substrates were commercial products of at least reagent grade.

Media and Solutions

YP+2% glucose medium was composed of 1% yeast extract, 2% peptone and 2% glucose.

PDA agar plates were composed of potato infusion (potato infusion was made by boiling 300 g of sliced (washed but unpeeled) potatoes in water for 30 minutes and then decanting or straining the broth through cheesecloth. Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, and 10 g of sodium chloride, and deionized water to 1 liter.

COVE sucrose plates were composed of 342 g of sucrose, 20 g of agar powder, 20 ml of COVE salt solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, 15 mM CsCl, Triton X-100 (50 μl/500 ml) were added.

COVE salt solution was composed bmf 26 g of MgSO₄·7H₂O, 26 g of KCL, 26 g of KH₂PO₄, 50 ml of COVE trace metal solution, and deionized water to 1 liter.

COVE trace metal solution was composed of 0.04 g of Na₂B₄O₇.10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionized water to 1 liter.

Dap-2C medium was composed of 20 g maltodextrin, 11 g MgSO4.7H2O, 1 g KH2PO4, 2 g Citric Acid, 5.2 g K3PO4.H2O, 0.5 g Yeast Extract (Difco), 1 ml Dowfax 63N10 (Dow Chemical Company), 0.5 ml KU6 trace metals solution, 2.5 g CaCO3, and deionized water to 1 liter. The medium was sterilized by autoclaving at 15 psi for 15 minutes (Bacteriological Analytical Manual, 8th Edition, Revision A, 1998). Before use, Dap-4C medium was added 3.5 ml sterile 50% (NH4)2HPO4 and 5 ml sterile 20% Lactic Acid per 150 ml medium.

KU6 trace metals solution was composed of 0.13 g NiCl2, 2.5 g CuSO4.5H2O, 13.9 g FeSO4.7H2O, 8.45 g MnSO4.H2O, 6.8 g ZnCl2, 3g Citric Acid, and deionized water to 1 liter.

MCS medium for growing the microbial test strains:

1.0% peptone, 0.8% egg extract, 0.4% yeast extract, 2.0% glucose, 0.5% sodium acetate trihydrate, 0.1% Tween 80, 0.2% dipotassium hydrogen phosphate, 0.2% triammonium citrate, 0.02% magnesium sulfate heptahydrate, 0.005% manganese sulfate tetrahyd rate

pH adjusted to 6.2, deionized water added to 1 liter.

MCS solid plates were made by adding 1.0% agar to the above formulation and autoclaving at 121° C. for 20 minutes before cooling and pouring the medium.

### Example 1

### Identification of the Didymella Exitialis Carbohydrate Oxidase Encoding Gene

IIlumina genomic sequencing of Didymella exitialis was performed according to standard procedure. Briefly, Genomic DNA was fractionated for 200-500 bp fragments and standard Illumina procdure was used to produce 100 bp paired end reads. 86,610,166 reads were obtained yielded 7,868,752,110 bp in total. GeneMark v2.3c was then used to identify 11344 genes. The DeCOx sequence was identified by performing a TFasty search against the nucleic acid sequnusing several known PFAM protein sequences as queries. Tfasty compares a protein sequence to a DNA sequence database, calculating similarities with frameshifts to the forward and reverse orientations, and allowing frame shifts within codons. Tfasty is part of the FASTA3 program suite (Pearson, 2000, Methods Mol. Biol. 132: 185-219). Multiple domains were identified on the protein DeCOx. The catalytic domain of DeCOx was identified by homology to the “C0G0277” module provided by NCBI, CDD (Conserved Domains, Marchler-Bauer A et al. (2013), “CDD: conserved domains and protein three-dimensional structure.”, Nucleic Acids Res. 41(D1):D384-52. The OR_GMC N and C domains defined from oxidoreductases active on Glucose, Methanol and Choline (Pfam; PF00732 and PF05199, in SEQ ID NO 1. These domains encompass the catalytic area of the enzyme involved in redox reactions (The Pfam protein families database: R. D. Finn, A. Bateman, J. Clements, P. Coggill, R. Y. Heger, K. Hetherington, L. Holm, J. Mistry, E. L. L. Sonnhammer, J. Tate, M. Punta Nucleic Acids Research (2014). COG0277 FAD/FMN-containing dehydrogenases. A region of the peptide N terminal to the oxidoreductase domain contains two carbohydrate binding domains belonging to the CBM18 family (Boraston A B, Bolam D N, Gilbert H J, Davies G J (2004) Carbohydrate-binding modules: fine-tuning polysaccharide recognition. Biochem. J. 382:769-781). Known properties of the CBM18 modules are interactions with chitin). The polypeptide coding sequence for the entire coding region was cloned from Didymella exitalis genomic DNA by PCR using the primers shown below (Example 3).

### Example 2

### Cloning and Expression of the Didymella Exitialis Carbohydrate Oxidase

The gene sequence (DNA positions 1 to 1948 in SEQ ID NO: 1) encoding the full length Didymella exitialis carbohydrate oxidase peptide (amino acid residues 1 to 632 in SEQ ID NO: 2) was identified and inserted into E.coli. Expression plasmids containing the insert were purified from the E. coli transformants, and transformed into an Aspergillus oryzae host cell. The transformed host cell was grown in liquid culture and the supernatant was harvested. The enzyme was purified by a combination of hydrophobic interaction chromatography, gel filtration and anion exchange chromatography.

### Example 2

### Didymella Exitialis Genomic DNA Extraction

Didymella exitialis was cultured in 500 ml Erlenmeyer shake flasks containing 100 ml YP+ 2% glucose. The cultures were shaken at 100rpm on a rotary shaker at 26 C for 4 days. Mycelia was harvested on Miracloth (Cat no. 475855-1R, Millapore Corp.) and frozen at −20 C until use.

The frozen mycelia was ground to a fine powder in a pre-chilled mortar with quartz sand and liquid nitrogen. The DNeasy Plant Mini kit by Qiagen was used to extract and purify the DNA from the mycelia (cat no. 69104, Qiagen Corp.).

### Example 3

### Construction of an Aspergillus oryzae Expression Vector Containing Didymella Exitialis Genomic Sequence Encoding an Oxidase Polypeptide

Two synthetic oligonucleotide primers shown below were designed to PCR amplify the Didymella exitialis DeCOx gene from the genomic DNA prepared in Example 2. An IN-FUSION™ Cloning Kit (Clontech, Mountain View, Calif., USA) was used to clone the fragment directly into the expression vector pDau109 (WO 2005/042735).

Primer-F
(SEQ ID NO: 3)
ACACAACTGGGGATCCACCATGAAACTCTTCCTCTCTCTCGCGG.
Primer-R
(SEQ ID NO: 4)
AGATCTCGAGAAGCTTACGCGCCAATGGCCTGCGG.

Bold letters represent the coding sequences. The underlined sequence is homologous to the insertion sites of pDau109.

Pfusion DNA polymerase (Cat. No. ML0530L from New England Biolabs was used for the fragment amplification. The NE Biolabs Pfusion protocol was used with the resulting mix for 50 uls

HF buffer (5×) 10 μL

Water 29.5 μL

dNTP (10 mM) 1 μL

Phusion pol. 0.5 μL

4 μL of Primer-F forward cloning primer at 2.5 μM concentration

4 μL of Primer-R reverse cloning primer at 2.5 μM concentration

1 μL gDNA

The reaction was placed in a BioRad Dyad PCR thermal cycler: running the following program:
2 min. 98° C.
15 sec. 98° C.
15 sec. 60° C.
60 sec. 72° C.
GoTo step2 35 times
5 min. 72° C.
Hold 10° C.

The reaction products were isolated by 1.0% agarose gel electrophoresis using 40 mM Tris base, 20 mM sodium acetate, 1 mM disodium EDTA (TAE) buffer where a 1068 bp product band was excised from the gel and purified using an illustra GFX® PCR DNA and Gel Band Purification Kit (GE Healthcare Life Sciences, Brondby, Denmark) according to the manufacturer's instructions. The fragment was then cloned into Hind III and Barn HI digested pDau109 using an IN-FUSION™ Cloning Kit resulting in plasmid pDeCOx. The treated plasmid and insert were transformed into Fusion Blue™ E. coli cells (Clontech, Mountain View, Calif., USA) according to the manufacturer's protocol and plated onto LB plates supplemented with 50 μg of ampicillin per ml. After incubating at 37° C. overnight, colonies were seen growing under selection on the LB ampicillin plates. Ten colonies transformed with the construct were cultivated in LB medium supplemented with 50 pg of ampicillin per ml and plasmid was isolated using a JETQUICK™ Plasmid Purification Spin Kit (GENOMED GmbH, Löhne, Germany) according to the manufacturer's instructions.

Cloning of the DeCOx gene into Hind III-Barn HI digested pDau109 resulted in the transcription of the Didymella exitialis DeCOx gene under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase.

The purified plasmid DNA of pDeCOx was transformed into protoplasts of Aspergillus oryzae MT3568, which were prepared according to the method of Christensen et al., 1988, Bio/Technology 6: 1419-1422. The selection plates consisted of COVE sucrose+10 mM acetamide+15 mM CsCl+TRITON® X-100 (50 μl/500 ml). The plates were incubated at 37° C. Eight transformants were selected and inoculated into separate wells of a 96 microtiter deep well plate (Nunc NS, Roskilde, Denmark) with each well containing 750 μl of YP+2% glucose medium or 750 μl of YP+2% maltodextrin. The plate was covered with Nunc pre-scored vinyl sealing tape (ThermoFisher Scientific, Roskilde, Denmark) and incubated at 26° C. stationary for 4 days. The colonies on the selection plates were also restreaked onto COVE sucrose (+10 mM acetamide +15 mM CsCI+TRITON® X-100 (50 μl/500 ml)). The plates were incubated at 37° C. and this selection procedure was repeated in order to stabilize the transformants. Several Aspergillus oryzae transformants produced the recombinant DeCOx oxidase of SEQ ID NO: 2 as judged by SDS-PAGE analysis.

### Example 4

### Construction of an Aspergillus oryzae Expression Vector Containing Fusarium graminearum Genomic Sequence Encoding the Carbohydrate Oxidase FgCOx

For comparative purposes, the Fusarium graminearum carbohydrate oxidase (FgCOx), which has also been reported to use GlcNAc as a substrate (Heuts et al, 2007. FEBS Lett. 581, 4905-4909), was cloned and expressed (SEQ ID NO: 5). The methodology for cloning and expression were as with the DeCOx cloning of example 3 with the following exceptions: The DNA was isolated from Fusarium graminearum PH-1 available from several sources including the Fungal Genetic Stock Center (Kansas City, Mo. 64110 USA).

The primers used in the PCR cloning of FgCOx were designed from the Fusarium graminearum whole genome shotgun sequence (The Broad Institute), protein encoding sequence: EMBL:ESU17750.1. The primers designed are as follows:

FgCOx-F
(SEQ ID NO: 6)
5′-ACACAACTGGGGATCCACCATGCATTTCAATACTTTGACATG.
FgCOx-R
(SEQ ID NO: 7)
5′-CTAGATCTCGAGAAGCTTCTAAGCCTTAGGCTTAATAGAC.

Bold letters represent coding sequence. The underlined sequence is homologous to the insertion sites of pDau109.

### Example 5

### Purification and Characterization of the FgCOx oxidase

Sterile filtered fermentation broth of FgCOx was washed by ultrafiltration using a PALL Ultrasette 10K cut-off suspended screen to obtain a conductivity below 3 mS. The pH of the sample was adjusted to 6 before being loaded onto a XK16 column with 20 ml SP Sepharose High Performance media pre-equilibrated in 25 mM MES, pH 6. The column was then washed with the buffer at 10 mL/min until a stable baseline was reached. The bound protein was eluted with a linear gradient from 0 to 0.5 M NaCl in 25 mM MES, pH 6 over 10 column volumes. Fractions of 10 ml were collected. The fractions containing pure oxidase, as estimated by SDS-PAGE, spectroscopy and activity assay, were pooled and concentrated. The concentrated solution of enzyme was stored at −20° C. until use. All purification steps were carried out at room temperature.

### Example 6

### Purification and Characterization of the DeCOx Oxidase

Two liters of culture fluid was prepared for the purification of the DeCOx enzyme. The strain, designated EXP9340, was streaked onto PDA plates and incubated at 34° C. for two weeks. Spores were removed in 5 ml of 0.01% TWEEN® 20 and 4 ml of spore suspension was inoculated into 3000 ml in all of DAP2C-1. 500 ml Erlenmeyer shake flasks were used with a medium volume of 100 ml per flask. The inncoulated shake flasks were incubated at 26° C. for 4 days with shaking at 150 rpm. The supernatants were harvested by filtering through a 0.22 μm filter.

The sterile filtered fermentation broth was made 1.2 M in ammonium sulphate and pH adjusted to 7.5 before being loaded onto a XK16 column with 20 mL Phenyl ToyoPearl media pre-equilibrated with 1.2 M ammonium sulphate in 25 mM Tris-HCl, pH 7.5. The column was then washed with 1.2 M ammonium sulphate at 10 mL/min until a stable baseline was reached.

The bound protein was eluted with 25 mM Tris-HCl (pH 7.5). Ten mL fractions were collected. The fractions of a bright yellow color, indicative of oxidase enzyme, were pooled. The pooled fractions were dialyzed against 25 mM Tris-HCl, pH 8.5 at 5° C. overnight. The dialyzed material was loaded onto a XK16 column with 20 ml Q Sepharose High Performance pre-equilibrated with 25 mM Tris-HCl, pH 8.5. The column was washed with the same buffer until a stable baseline was reached. The bound protein was eluted with a linear gradient from 0 to 0.5 M NaCl in 25 mM Tris-HCl, pH 8.5 over 15 column volumes. Fractions of 10 mL were collected. The fractions containing pure oxidase, as estimated by SDS-PAGE, spectroscopy and activity assay, were pooled and concentrated. The concentrated solution of enzyme was stored at −20° C. until use. All purification steps, apart from dialysis, were carried out at room temperature.

The enzymatic activity was determined spectrophotometrically using a coupled assay with peroxidase for detection of hydrogen peroxide generated by the oxidase reaction. Assays were carried out at room temperature in 96 well microtiter plates containing 100 μl 0.1 M Britton-Robinson buffer (pH 3-10), 20 μl 0.1 M carbohydrate substrate, 20 μl 6 mM 4-amino antipyrine, 20 μl 15 mM TOPS, 40 PODU/ml rCiP. The reaction was started by adding 20 μl oxidase solution diluted to 0.05 g/L. Absorption is measured at 550 nm as a function time over 5 minutes using the Vmax microliter plate ready from Molecular Devices and the activity is taken as the slope of the linear increase

TABLE 1
Enzymatic activity towards various substrates.

| | | | Measured rate | | pH range with >50% | |
|---|---|---|---|---|---|---|
| | pH optimum | | at pH optimum | | of max rate | |
| | DeCOx | FgCOx | DeCOx | FgCOx | DeCOx | FgCOx |
| N-acetyl-D-glucosamine | 9 | 6 | 258.9 | 7.0 | 8-9 | 4-9 |
| N-acetyl-D-galactosamine | 9 | 0 | 110.7 | 0 | 8-10 | 0 |
| L+-arabinose | 9 | 10 | 0.2 | 0.2 | 6-8 | 5-10 |
| D+-cellobiose | 9 | 8 | 78.4 | 2.4 | 9-10 | 8 |
| 2-Deoxy-D-glucose | 9 | 10 | 3.2 | 0.5 | 9-10 | 10 |
| D-fructose | 5 | 10 | 0.8 | 0.4 | 5 | 10 |
| D+-galactose | 9 | 7 | 1.6 | 0.2 | 9 | 6-10 |
| D+-glucose | 9 | 10 | 3.5 | 0.4 | 9-10 | 9-10 |
| D-Lactose | 9 | 3 | 43.7 | 1.6 | 9-10 | 3 |
| Lactulose | 10 | 10 | 0.8 | 5.8 | 9-10 | 10 |
| D-Maltose | 9 | 10 | 48.3 | 1.6 | 9-10 | 9-10 |
| D-mannitol | 9 | 8 | 0.2 | 0.2 | 5-9 | 6-10 |
| D-mannose | 9 | 10 | 1.1 | 0.4 | 9-10 | 8-10 |
| D-raffinose | 9 | 10 | 1.2 | 0.3 | 8-10 | 5-10 |
| L-sorbose | 8 | 7 | 0.2 | 0.2 | 5-10 | 4-10 |
| Sucrose | 4 | 9 | 1.3 | 0.1 | 4 | 9 |
| D+-xylose | 7 | 9 | 0.7 | 0.3 | 7 | 9 |

### Example 7

### Antimicrobial Activity of the Didymella exitialis Oxidase, RDA

The antimicrobial activity of DeCOx against S. carnosus and E. coli was tested using an radial diffusion assay (RDA) as described previously by Lehrer et al. (Lehrer R I, Rosenman M, Harwig S S et al. (1991), “Ultrasensitive assays for endogenous antimicrobial polypeptides”, J Immunol Methods, 137:167-73), but with several modifications. Inoculum of S. carnosus (ATCC 51365) or E. coli (ATCC 10536) from freeze stocks was streaked on Tryptone Soya Agar (TSA) (Oxoid, CM 131) plates and incubated overnight at 37° C. Colonies were suspended in 0.9% NaCl and the suspensions were adjusted to McFarland std 1 (1.0 ml BaCl2 (1.175%)+99.0 ml H2SO4 (1%), turbidity equivalent to 3×10e8 CFU/ml). 87% sterile glycerol was added to the final concentration of 20% and cells were frozen at −80° C. until use. 10-fold dilution series were prepared of the freeze stocks in 0.9% NaCl and 100 pl of the dilutions were plated on Tryptone Soya Agar (TSA) (Oxoid, CM 131) plates for estimation of colony forming units per milliliter. When preparing RDA plates 30 mL of melted 1/10 Mueller-Hinton broth (MHB) (Sigma/Fluka, 90922) with 1% agarose and was cooled to 40° C., supplemented to around 5.0×10⁵ cfu/mL with S. carnosus or E. coli and N-acetyl-D-glucosamine (Sigma Aldrich A4106) (GlcNAc) added before pouring into a single-well Omnitray (Nunc) plate. The Omnitray plate was covered with a TSP lid (Nunc) with 96 attached pins and left to solidify. The TSP lid was removed; leaving 96 wells, in which 10 μL of the compound of interest could be tested.

10 μl of the test solutions were spotted per well and the plates were incubated over night at 37° C. The following day a clearing zone indicated no growth of test bacteria thereby indicating antimicrobial activity. The clearing zones were visualized by coloring with MTT (3-(4,5-dimethylthiazol-2-yl)-2,5-diphenyltetrazolium bromide, a yellow tertrazole), that is reduced to purple formazan in living cells (Mosmann, Tim (1983), “Rapid colorimetric assay for cellular growth and survival: application to proliferation and cytotoxicity assays”, Journal of Immunological Methods 65 (1-2): 55-63). This coloring provides for a dark coloring of living cells and no coloring of the clearing zones without living cells.

10 μl gentamicin (100 μg/ml) was included as a positive control. Growth inhibition was observed as listed in table 2.

TABLE 2
Antimicrobial activity of DeCOx tested against S. carnosus
and E. coli in the presence of GlcNAc

| | S. carnosus | | | | | | E. coli | | | | | |
|---|---|---|---|---|---|---|---|---|---|---|---|---|
| DeCOx μg/ml | 10 | 100 | 133 | 200 | 400 | GEN | 10 | 100 | 133 | 200 | 400 | GEN |
| 1 mM GlcNAc | ++ | ++ | ++ | ++ | +++ | +++ | − | + | + | + | + | ++ |
| 10 mM GlcNAc | ++ | +++ | +++ | +++ | +++ | +++ | + | ++ | ++ | ++ | ++ | ++ |
| 50 mM GlcNAc | +++ | ++++ | ++++ | ++++ | ++++ | +++ | + | ++ | ++ | ++ | ++ | ++ |

+ zone diameter ≧5 and <15 mm,
++ zone diameter ≧15 and <25 mm,
+++ zone diameter ≧25 and <40 mm,
++++ zone diameter ≧40 mm
GEN = gentamicin

The RDA assay showed that in the presence of only 1 mM GlcNAc a significant anti-microbial activity of DeCOx against S. carnosus was detected. Inoculation of 10 μl of 10-400 μg/ml DeCOx prevented growth of S. carnosus for all concentrations tested and thus resulted in large clearing zones. The size of the clearing zone increased in proportion to a higher concentration of GlcNAc and DeCOx added. For E. coli growth inhibition was observed in the presence of 10 and 50 mM GlcNAc and for all concentrations of DeCOx tested (10-400 μg/ml). When 1 mM GlcNAc was added in the E. coli radial diffusion assay, only very small clearing zones were observed around the inoculum (<10 mm). Thus, S. carnosus (ATCC 51365) is more sensitive to the anti-microbial activity of DeCOx than E. coli (ATCC 10536).

### Example 8

### Bactericidal Effect of the Didymella exitialis Oxidase

S. carnosus (ATCC 51365) and E. coli (ATCC 10536) were inoculated into Mueller Hinton Broth (MHB) (Sigma/Fluka, 90922) to around 5.0×10⁵ cfu/mL. Bacteria were grown at 37° C. in MHB, MHB added 10 mM N-acetyl-D-glucosamine (GlcNAc), MHB added 100 μg/ml DeCOx or MHB added 10 mM GlcNAc and 100 μg/ml DeCOx. Samples were withdrawn at time 0, 2, 4 and 24 h and 10-fold dilution series to 10⁻⁵ in 0.9% NaCl were made. 100 μl from each dilution series were plated on Tryptone Soya Agar (TSA) (Oxoid, CM 131) plates and incubated at 37° C. overnight.

TABLE 3
Bactericidal effect of the Didymella exitialis oxidase

| | DeCOx | GlcNAc | | | | |
|---|---|---|---|---|---|---|
| | 100 | 10 | cfu/ml | | | |
| Strain | μg/ml | mM | 0 h | 2 h | 4 h | 24 h |
| S. carnosus | − | − | 9E+05 | 1E+06 | 2E+06 | 1E+08 |
| S. carnosus | − | + | 8E+05 | 1E+06 | 3E+06 | 2E+08 |
| S. carnosus | + | − | 8E+05 | 9E+05 | 8E+06 | 2E+08 |
| S. carnosus | + | + | 7E+05 | 7E+05 | 2E+05 | <1E+02 |
| E. coli | − | − | 5E+05 | 7E+05 | 4E+07 | 4E+08 |
| E. coli | − | + | 6E+05 | 7E+05 | 4E+07 | 5E+08 |
| E. coli | + | − | 5E+05 | 6E+05 | 6E+07 | 5E+08 |
| E. coli | + | + | 6E+05 | 2E+05 | 4E+04 | <1E+02 |

CFU counts showed that both S. carnosus and E. coli are killed in the presence of GlcNAc and DeCOx under the tested conditions. After 24 h incubation the amount of cells was below the detection limit of 100 cfu/ml for both strains. In contrast, neither the presence of DeCOx or GlcNAc alone had any influence on the growth of the two strains.

### Example 9

### Fungal Sporicidal Assay

The two fungal strains Aspergillus carbonarius BR00732 (CBS 139193) and Aspergillus niger strain BR00883 (accession number CBS 139194) were grown on PDA medium petri plates at 26° C. and allowed to sporulate for several days. 5 ml dionized water with 0.1% Tween80 was added to the plate and the spores were suspended in the solution then transferred to Falcon 2059 tubes. Glycerol was added to 10% as a cryopreservent and the spore suspensions were frozen in aliquots at −20° C. until further use. Cove-N agar with 1% glucose and and Cove-N ager with 1% glucose and 100 mM GlcNAc were prepared in 51 mm Petri plates, 5 ml per plate.

The purified DeCOx enzyme prepared in the following buffer: 25 mM Tris pH 8,5 200 mM NaCl. The concentration was 10.48 mg/l or 163.47 μM as estimated by the OD 280 calculation method.

100 μM stock of DeCOx enzyme was made by dilution in liquid Cove-N medium. 100 μl of this stock was applied to 51 mm petri plates described above. The solutions were spread evenly with a sterile glass Drigalski spatula. The enzyme was allowed to diffuse in to the medium for three hours before applying the fungal spores. The fungal spore suspensions in 10% glycerol were evaluated for viable spores per ml by plating dilutions of a frozen spore stock on PDA medium at various dilutions in distilled water. A spore dilution of 500-1000 spores per ml was used and 100 μl of this dilution plated on to the enzyme treated plates. The plates were then incubated at 30° C. for two days.

TABLE 4
Fungicidal effect of DeCOx.

| Sample | GlcNAc | A. niger | A. carb | DeCOx | Colonies |
|---|---|---|---|---|---|
| 1a | − | + | − | + | 16 |
| 1b | + | + | − | + | 0 |
| 2a | − | − | + | + | 94 |
| 2b | + | − | + | + | 0 |

1= Aspergillus niger,
2= Aspergillus cabinarius

The data shows that the DeCOx enzyme in combination with N-acetyl-N-glucosamine is sporicidal or renders the spores of the two fungi incapable of germinating under the conditions tested.

### Example 10

### Selective Control of Lactobacillus reuteri Under Anaerobic Conditions

A brewer's yeast strain and a Lactobacillus reuteri strain were used. Saccharomyces cereviseae CBS1171 is available from Centraalbureau voor Schimmelcultures, Ultrect, Netherlands. Lactobacillus reuteri DSM20016 is available from The Leibniz Institute DSMZ−German Collection of Microorganisms and Cell Cultures, Braunschweig, Germany.

Microaerobic condtions, in which very little oxygen is available, were achieved with an Oxoid CampyGen (no.CN0025) in an Oxoid 2.5 liter AnaeroJar (no. AG0025).

The DeCOx purified enzyme sample were prepared as in Example 9. 2/4strength MRS medium with 5 mM N-acetyl-D-glucosamine (Sigma Aldrich A4106) was prepared by diluting full strength MRS medium with 0.9% NaCl.

Saccharomyces cereviseae starter culture was prepared by suspending Saccharomyces cereviseae cells from a fresh petri plate culture (PDA plates, 30° C. incubation 24 hours) in 0.9% NaCl solution to an OD480 of approximately 1.0.

Lactobacillus reuteri starter culture was prepared by inoculating the bacteria in 10 ml MRS medium in a Falcon 2059, 12 ml plastic tube and sealing the cap with Parafilm™ (Fischer Scientific, Denmark). The tube was incubated stationary at 30 degrees ° C. for 24 hours. 200 μl of ¾MRS medium (with or without 5mM GlcNAc), was added into a sterile NUNC 96 Deepwell culture plates (Nunc 278752). Final concentrations of enzyme, from 0, 6.35, 12.7, 25.4, 50.8 or 101.6 μM were tested in the indicated wells. Lastly, 10 μl of either the yeast or Lactobacillus suspensions were inoculated into the Deep well culture plates (Nunc 278752) according to the scheme shown below (Table 5). The plate was placed in a 2.5 liter Oxoid Anaero jar and a CampyGen system was added before sealing the vessel. The Anaero jar was incubated at 30 degrees ° C. for 24 hours. The cells in the deep well plate were resuspended by shaking on a for app. 1 min., 100 μl were transferred to flat bottom MTW; COSTAR 3635 microtiter plate and measured at OD 480 on a VersaMax microplate reader (Molecular Devices Inc). OD480 values shown in Table 5 are corrected values in which the MRS background has been subtracted.

TABLE 5
Effect of DeCOx on yeast and Lactobacillus

| Treatment | Yeast | Lacto. | GlcNAc | DeCOx | OD 480 |
|---|---|---|---|---|---|
| 1 | + | − | − | 0 | 0.66 |
| 2 | + | − | + | 0 | 0.63 |
| 3 | − | + | − | 0 | 0.93 |
| 4 | − | + | + | 0 | 0.99 |
| 5 | + | − | − | 6.35 | 0.72 |
| 6 | + | − | + | 6.35 | 0.59 |
| 7 | − | + | − | 6.35 | 1 |
| 8 | − | + | + | 6.35 | 0.1 |
| 9 | + | − | − | 12.7 | 0.7 |
| 10 | + | − | + | 12.7 | 0.59 |
| 11 | − | + | − | 12.7 | 1.2 |
| 12 | − | + | + | 12.7 | 0.07 |
| 13 | + | − | − | 25.4 | 0.72 |
| 14 | + | − | + | 25.4 | 0.52 |
| 15 | − | + | − | 25.4 | 0.87 |
| 16 | − | + | + | 25.4 | 0.09 |
| 17 | + | − | − | 50.8 | 0.64 |
| 18 | + | − | + | 50.8 | 0.64 |
| 19 | − | + | − | 50.8 | 0.7 |
| 20 | − | + | + | 50.8 | 0.14 |
| 21 | + | − | − | 101.6 | 0.73 |
| 22 | + | − | + | 101.6 | 0.67 |
| 23 | − | + | − | 101.6 | 0.78 |
| 24 | − | + | + | 101.6 | 0.21 |

The Lactobacillus reuteri strain is strongly affected by the DeCOx enzyme in the presence of N-acetyl-D-glucosamine. Yeast, on the other hand, appears not to be as strongly affected by the combined effect of the DeCOx enzyme and N-acetyl-D-glucosamine. Furthermore, the amount of enzyme needed in order to achieve a depression of the Lactobacillus growth, as judged by depression of the optical density at OD 480, is in the range of between 6.35 to over 100 μM DeCOx.

1-15. (canceled)

16. A polypeptide having N-acetyl-D-glucosamine oxidase activity, selected from the group consisting of:
(a) a polypeptide having at least 75% sequence identity to the mature polypeptide of SEQ ID NO: 2;
(b) a polypeptide encoded by a polynucleotide that hybridizes under high stringency conditions with (i) the mature polypeptide coding sequence of SEQ ID NO: 1, (ii) the cDNA sequence thereof, or (iii) the full-length complement of (i) or (ii);
(c) a polypeptide encoded by a polynucleotide having at least 75% sequence identity to the mature polypeptide coding sequence of SEQ ID NO: 1 or the cDNA sequence thereof; and
(d) a fragment of the polypeptide of (a), (b), or (c) that has N-acetyl-D-glucosamine oxidase activity.

17. The polypeptide of claim 16, comprising or consisting of SEQ ID NO: 2 or the mature polypeptide of SEQ ID NO: 2.

18. The polypeptide of claim 16, wherein the mature polypeptide is amino acids 20 to 632 of SEQ ID NO: 2.

19. The polypeptide of claim 16, which has at least 20% of the antimicrobial activity of the mature polypeptide of SEQ ID NO: 2.

20. The polypeptide of claim 19, wherein the antimicrobial activity is assessed using a method described in Example 6, 7, 8, or 9.

21. The polypeptide of claim 19, wherein the antimicrobial activity is assessed as fungicidal or sporicidal effect towards any of the fungal strains Aspergillus carbonarius BR00732 (CBS 139193) and Aspergillus niger strain BR00883 (accession number CBS 139194).

22. A cleaning composition consisting of the polypeptide of claim 16 and one or more ingredients used in cleaning compositions, such as a surfactant.

22. A method of cleaning and/or disinfecting comprising applying the composition of claim 21.

24. The method of claim 22, which is cleaning-in-place.

25. A polynucleotide encoding the polypeptide of claim 16.

26. A nucleic acid construct or expression vector comprising the polynucleotide of claim 25, wherein the polynucleotide is operably linked to one or more control sequences that direct the production of the polypeptide in a host cell.

27. A recombinant host cell comprising the polynucleotide of claim 25, wherein the polynucleotide is operably linked to one or more control sequences that direct the production of the polypeptide in the host cell.

28. A method of producing a polypeptide having N-acetyl-D-glucosamine oxidase activity, comprising cultivating the host cell of claim 27 under conditions conducive for production of the polypeptide.

29. The method of claim 28, further comprising recovering the polypeptide.


SEQUENCE LISTING (7 sequences)

SEQ ID NO: 1 - DNA, 1948 bp, Didymella exitialis
  CDS (1)..(141)
  misc_feature (1)..(57): Sig_peptide
  misc_feature (58)..(1945): Mat_peptide
  misc_feature (82)..(144): Binding domain
  Intron (142)..(190)
  CDS (191)..(1945)
  misc_feature (350)..(481): Binding domain
  misc_feature (509)..(1945 ): Catalytic domain
atgaaactcttcctctctctcgcggcgagcgccctcgctttcggggctgtgaccgctgcgactgtctcgccagatggctcgtgtgctggtactagcaagtacacctgtgcgggtagtggctatggcaactgttgttcgcaggtaagtttctgcccctcaccccatctgtgttggaaactgattcttacagtatggttggtgtggttcttcagacgcccactgtaaggctggctgcaactctgcttttggcacctgtgccggcgctgcttcttcgactttgtctacccgttcctcgactcgcctagctccttcgcctacaccgtccaagattgtcaccccggatgctacatgtggtggtagcaaaggctacacatgcgctggcagctcgttcggaaactgctgcagcagcagcgggtactgcggcactactaatgcctactgcggatcagggtgccagtctgcattcgggagctgtggcaatggtggcaccgtcacgagcaaaactacctctgcttctgcttctgcgtctgcgaccccgtctggctcggtccttcagtgcttgaatggaaagaatgtgccgtacaagatgacgtctgacggcgagtacgacgcgcttgtgaggccttacaatctcgccatctcgttcaagccatccgtggtcgtgctcccgcagacccagcagaacatccaggacgctgtcgtatgcgccgggcagtctggcctcaaggtccaggccaagtcgggcggccattcttacgccagcttcagctctggcggtaaagacggctccatgatgatcagcctccagacgtttcagaaggtcgagctcaacgccaacaccggcattgccaaggtcggtggtggtgtgcgtctcggaaacctcgctgatggcatctatactcaaggccagaagggtctgtctcacggaacttgccctggtgtcggaatcggaggccacttcacgcacggcggctacggccacacatcgcggcattggggtcttgccatggatcagatcgtgtctgcggacgttgtgctggcagatggctccctcgtgacagcatcagcaacacagaacagtgaaattttctgggcgatccggggcgccgccgactcgttcggtatcgtgaccaacttctacctgcagacacaagcagcaccctcgagcatcacatactttgcgtttgcattcccagccgtatggaacacgaaggcgaccttcaccaactcgttcctgcacatccaggacttcgccaccaacgcgagtgtcattgacaaccgcatctcattcggcatctacatggacaactacggcacctacagcctcagtggcgcctttttcggctccgtcgacgagttcaactcgaagatcaagcccgagctcctccgcacgctccccacaccaacggaccccaccgtcaaagcctacagctgggtcgactacctcgtcctcgtatccggcaagaacacgatcaaagtcccactgaccaactacgacgaccacgaagacttctttgcgaaatccatcacagtccccgagtcgaccggcctcacagcatcggccctcaacgccttctacgacaaagtccgcggcacaagcaccgagttcttcaccatcatcaacctatacggtggacccggcagcgccatcaacgcgcgcgacaccgactttgcggcctactcggaccgcgacagcctgtgggtgttccagaactacggatacacgtcgtcgacaaaggatttcgtcaacggcatcaacagcgccattatcaacgcgcagccacagacggcgtttggcgcgtaccttaactacgtcgatccctcgtatgatgcagcgacggcgcatcggctgtactatggtgatgcgttgtatgcgcgtcttgctgcactgaagaagaaggtcgatcccaaggcggtcttttggaacccgcaggccattggcgcgtag

SEQ ID NO: 2 - protein, 632 aa, Didymella exitialis
MKLFLSLAASALAFGAVTAATVSPDGSCAGTSKYTCAGSGYGNCCSQYGWCGSSDAHCKAGCNSAFGTCAGAASSTLSTRSSTRLAPSPTPSKIVTPDATCGGSKGYTCAGSSFGNCCSSSGYCGTTNAYCGSGCQSAFGSCGNGGTVTSKTTSASASASATPSGSVLQCLNGKNVPYKMTSDGEYDALVRPYNLAISFKPSVVVLPQTQQNIQDAVVCAGQSGLKVQAKSGGHSYASFSSGGKDGSMMISLQTFQKVELNANTGIAKVGGGVRLGNLADGIYTQGQKGLSHGTCPGVGIGGHFTHGGYGHTSRHWGLAMDQIVSADVVLADGSLVTASATQNSEIFWAIRGAADSFGIVTNFYLQTQAAPSSITYFAFAFPAVWNTKATFTNSFLHIQDFATNASVIDNRISFGIYMDNYGTYSLSGAFFGSVDEFNSKIKPELLRTLPTPTDPTVKAYSWVDYLVLVSGKNTIKVPLTNYDDHEDFFAKSITVPESTGLTASALNAFYDKVRGTSTEFFTIINLYGGPGSAINARDTDFAAYSDRDSLWVFQNYGYTSSTKDFVNGINSAIINAQPQTAFGAYLNYVDPSYDAATAHRLYYGDALYARLAALKKKVDPKAVFWNPQAIGA

SEQ ID NO: 3 - DNA, 44 bp, Artificial Sequence
  Primer-F
acacaactggggatccaccatgaaactcttcctctctctcgcgg

SEQ ID NO: 4 - DNA, 35 bp, Artificial Sequence
  Primer-F
agatctcgagaagcttacgcgccaatggcctgcgg

SEQ ID NO: 5 - protein, 492 aa, Fusarium graminearum
  MISC_FEATURE (1)..(19): Signal peptide
  MISC_FEATURE (20)..(492): Mature peptide
MHFNTLTCVLVGLVAHTSAVPTKREAVNSCLTQAKVPTDAQGSQSWKEDGTAYNLRLPFEPAAIAVPTTVAQVSAAVECGAKHGVAISAKSGGHSYTSLGFGGEDGHLMIELDRMYSVKLAKDGTAKIQPGARLGHVATELWNQGKRALAHGTCPGVGLGGHALHGGYGMVARKHGLTLDLMIGATVVLPTGKVVHCSKTENSDLFWGIRGAGANFGVVVELEFQTFAAPEKITYFDIGLNWDQNTAPQGLYDFQEFGKGMPAEITMQMGVSKNGYSVDGAYIGDEASLRKALQPLVQKFGGVQVTATTVDWMGLVTHFAGAGVNVNPTSASYDAHDNFYASSLAAPALTLAEFKSFVNFVSTTGKSSSHSWWLQMDITGGTYSAVSKPKPSDTAYVHRDTLLLFQFYDSVAATAQYPSDGFNLIKGLRQSISSSLKAGTWGMYANYPDSQIKNDRATEMYWGSNVAKLEAVKAKYDPKNLFRNPQSIKPKA

SEQ ID NO: 6 - DNA, 42 bp, Artificial Sequence
  Primer-F
acacaactggggatccaccatgcatttcaatactttgacatg

SEQ ID NO: 7 - DNA, 40 bp, Artificial Sequence
  Primer-R
ctagatctcgagaagcttctaagccttaggcttaatagac